(19) United States
(12) Patent Application Publication
(10) Pub. No.: US 2024/0050470 A1
(43) Pub. Date: Feb. 15, 2024

(54) Method for Inhibiting Reperfusion Injury

(71) Applicants: Corvitus LLC, Palm Beach Gardens, FL (US); Corvitus GmbH, Marktring (AT)

(72) Inventors: Tracy Goeken, Palm Beach Gardens, FL (US); Bernard Winkler, Vienna (AT)

(21) Appl. No.: 18/489,703

(22) Filed: Oct. 18, 2023

Related U.S. Application Data
(63) Continuation-in-part of application No. PCT/US2022/072069, filed on May 3, 2022.

(30) Foreign Application Priority Data
May 3, 2022 (WO) PCT/US2022/072069

(51) Int. Cl. A61K 33/42; A61K 33/06; A61K 38/06; A61K 33/14; A61K 31/7004; A61K 31/375; A61K 31/198; A61K 31/205; A61P 9/10; A61K 9/00

(52) U.S. Cl. CPC A61K 33/42; A61K 33/06; A61K 38/063; A61K 33/14; A61K 31/7004; A61K 31/375; A61K 31/198; A61K 31/205; A61P 9/10; A61K 9/0019

(57) ABSTRACT

A method to inhibit reperfusion injury to a tissue that has experienced ischemia comprising treating the ischemic tissue with an antioxidant solution prior to or in conjunction with reperfusion of blood into the ischemic tissue pursuant to artery bypass surgery, angioplasty or in conjunction with an angiogram. The antioxidant solution is comprised of water, dissolved salts of calcium, potassium, magnesium, sodium, one or more buffers, L-Arginine, glucose, glutathione, and ascorbic acid and wherein the solution is ionically and pH balanced to a physiologic pH, preferably 7.2-7.4.

Related Applications: The present application is a continuation-in-part of and claims priority to International Patent Application No. PCT/US2022/072069 filed on 3 May 2022, which claims priority to U.S. Provisional Application Ser. No. 63/201,530 filed 4 May 2021 and to U.S. Provisional Application Ser. No. 63/201,534 filed 4 May 2021. The teachings of the applications are incorporated herein in their entirety by reference.

### BACKGROUND OF THE INVENTION

Reperfusion injury, sometimes called ischemia-reperfusion injury (IRI) or reoxygenation injury, is the tissue damage caused when blood supply returns to tissue after a period of ischemia or lack of oxygen (anoxia or hypoxia). This can result in the induction of oxidative stress, through the overproduction of reactive oxygen species (ROS) such as superoxide O₂⁻ and through inflammation in general. Ischemia can occur in an organ or in tissue as a result of stenosis in a blood vessel. Stenosis is an abnormal narrowing of a conduit in the body, especially in a vein or artery such as that which occurs in an ischemic stroke or a myocardial infarction. Reperfusion of ischemic tissues is often associated with microvascular injury, particularly due to increased permeability of capillaries and arterioles that lead to an increase in diffusion and fluid filtration across the tissues. Activated endothelial cells produce more reactive oxygen species but less nitric oxide following reperfusion, and the imbalance results in a subsequent inflammatory response.

The inflammatory response is partially responsible for the damage of reperfusion injury. White blood cells, carried to the area by newly returning blood, release a host of inflammatory factors such as interleukins as well as free radicals in response to tissue damage. The restored blood flow reintroduces oxygen with cells that damages cellular proteins, DNA and the plasma membrane. Damage to the cell's membrane may in turn cause the release of more free radicals. Such reactive species may also act indirectly in redox signaling to turn on apoptosis. White blood cells may also bind to the endothelium of small capillaries, obstructing them and leading to more ischemia.

The heart can be damaged in different ways including acute coronary syndrome, myocardial infarction, myocarditis and by trauma. Reperfusion injury also plays a major part in the biochemistry of hypoxic brain injury in stroke. Similar failure processes are involved in brain failure following reversal of cardiac arrest. Repeated bouts of ischemia and reperfusion injury are a factor leading to the formation and failure to heal of chronic wounds such as pressure sores and diabetic foot ulcer.

There is a need for a method to reduce injury to tissues caused by reperfusion after an ischemic event. Such an ischemic event can occur in acute coronary syndrome, myocardial infarction, cerebral ischemic stroke, pulmonary embolism and by trauma of various tissues.

### SUMMARY OF THE INVENTION

The present invention fills this need by providing for a method to inhibit reperfusion injury to a tissue that has experienced ischemia comprising treating the tissue prior to or in conjunction with reperfusion of blood into the ischemic tissue with an antioxidant solution. The antioxidant solution is comprised of a physiologic salt solution, which is ionically balanced, pH balanced to a physiologic pH, preferably 7.2-7.4, contains ascorbic acid, glutathione and arginine as a substrate for nitric oxide synthase. The physiologic salt solution is comprised of water, dissolved salts of calcium, potassium, magnesium, sodium and one or more buffers. In an alternative embodiment, the antioxidant solution is further comprised of creatine and/or carnitine. In a preferred embodiment the physiologic salt solution consists essentially of water, dissolved salts of calcium, potassium, magnesium, sodium and one or more buffers.

In one embodiment in artery bypass grafting surgery, such as coronary artery bypass grafting (CABG) or cerebral artery bypass grafting surgery, the antioxidant solution is flushed through a bypass conduit such as through a section of the saphenous vein that has been anastomosed to one of the arteries downstream of an occlusion within the artery. The anastomosis is distal or downstream from the occlusion/stenosis within the artery. The flushing of the conduit with the antioxidant solution occurs prior to the anastomosis of the conduit to a blood-providing arterial vessel such as the aorta or branch of the aorta in the heart or to, for example, the carotid artery in the neck for cerebral artery bypass surgery. This flushing administers the antioxidant solution to the section of the heart or the brain that has been deprived of blood due to the occlusion/stenosis prior to blood-flow being restored to the ischemic tissue by anastomosis of the opposite end of the conduit to a blood-providing blood vessel such as the aorta or branch of the aorta in the heart or carotid artery or occipital artery for brain bypass surgery.

In another embodiment, the antioxidant solution is flushed through an artery that has an occlusion and that is undergoing angioplasty prior to the angioplasty and/or in conjunction with the angioplasty. Using a catheter having a needle, the needle pierces the occlusion, and the antioxidant solution is injected into the portion of the artery downstream from the blockage prior to the blood-flow being restored. This reduces the damage that can be caused by reperfusion of blood into ischemic tissue because the solution scavenges the reactive oxygen species and other inflammatory factors that occur when blood-flow is restored.

In another embodiment, one or more arteries are flushed with the antioxidant solution in conjunction with angiography or arteriography of the arteries. In another embodiment, the solution is flushed through the right or left coronary arteries to perfuse the heart with the solution prior to or after coronary bypass surgery, angioplasty or an angiogram with the proviso that the solution is not used as a cardioplegic solution. This inhibits ischemia/reperfusion injury as is evidenced by the decrease in troponin levels that are release by the heart as a result of ischemia/reperfusion.

In another embodiment, the solution is administered distal from a blockage in an artery of the brain prior to removal of the blockage in the artery and prior to the reintroduction of blood into the ischemic portion of the brain thus inhibiting reperfusion injury.

In another embodiment, the antioxidant solution is used in conjunction with peripheral vascular bypass grafting surgery. A peripheral vascular bypass is a procedure performed to reroute blood flow around a blocked or occluded artery. It does so by creating a new pathway for blood to flow using a conduit such as a synthetic graft (plastic tube) or a vein harvested from the patient such as a saphenous vein. The antioxidant solution is used to inhibit reperfusion injury by infusing the antioxidant solution through the bypass conduit after the conduit has been anastomosed to the artery downstream or distal from the occlusion and prior to anastomoses of the conduit onto the blood-providing artery. This infusion inhibits reperfusion injury of the ischemic tissue downstream of the occlusion.

### DESCRIPTION OF THE DRAWINGS

FIG. 1 is a general schematic of a heart including the coronary arteries.

FIG. 2 shows the flushing of a section of a saphenous vein (40) one end of which (42) has been anastomosed to the left coronary artery (30) downstream from an infarction/blockage/stenosis (36).

FIG. 3 shows the saphenous vein anastomosed to the aorta at the proximal end of the saphenous vein and to the coronary artery at the distal end of the saphenous vein.

FIG. 4 shows a catheter that is delivering the anti-oxidant solution to the right and left coronary arteries.

FIGS. 5A-5C show the delivery of the antioxidant solution in conjunction with angioplasty or angiography.

### DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

As used herein, the term “patient” includes members of the animal kingdom including but not limited to human beings. As used herein, the term “cardioplegia or cardioplegic solution is a solution” refers to a solution to bring about asystole/arresting/stopping or heart paralysis in association with cardiac surgery.

As used herein, an “antioxidant” is a substance that, when present in a mixture or structure containing an oxidizable substrate biological molecule, delays or prevents oxidation of the substrate biological molecule. For example, ascorbic acid and glutathione are antioxidants.

A “Balanced salt solution” is defined as an aqueous solution that is osmotically balanced to prevent acute cell or tissue damage.

A “Buffered salt solution” is defined as a balanced salt solution to which chemicals have been added to maintain a predetermined physiological pH range.

A bypass conduit” is defined as a surgically installed alternate route for the blood to bypass an obstruction.

A Catheter is a flexible tube inserted through a narrow opening into a body cavity.

A “Cellular reducing agent” is defined as a substance that loses electrons easily thereby causing other substances to be reduced chemically.

An Embolus is a blood clot, air bubble, piece of fatty deposit or other object that has been carried in the bloodstream to lodge in a vessel and cause an embolism.

An Embolism is an obstruction in a blood vessel due to a blood clot or other foreign matter that gets stuck while traveling through the bloodstream.

An Infarct is an area of necrosis in a tissue or organ resulting from obstruction of the local circulation by a thrombus or embolus.

Reperfusion is the action of restoring the flow of blood to an organ or tissue.

A Stenosis, blockage or occlusion is the abnormal narrowing of a passage in the body.

A Thrombus is a blood clot formed in situ within the vascular system of the body and impeding blood flow.

Angiography or arteriography is a medical imaging technique used to visualize the inside, or lumen, of blood vessels and organs of the body, with particular interest in the arteries, veins, and the heart chambers. Depending on the type of angiogram, access to the blood vessels is gained most commonly through the femoral artery to look at the left side of the heart and at the arterial system; or the jugular or femoral vein, to look at the right side of the heart and at the venous system. Using a system of guide wires and catheters, a type of contrast agent (which shows up by absorbing the X-rays), is added to the blood to make it visible on the X-ray images.

Myocardial infarction (MI) refers to tissue death (infarction) of the heart muscle (myocardium) caused by ischemia, that is lack of oxygen delivery to myocardial tissue. It is a type of acute coronary syndrome, which describes a sudden or short-term change in symptoms related to blood flow to the heart. Unlike the other type of acute coronary syndrome, unstable angina, a myocardial infarction occurs when there is cell death, this can be estimated by measuring by a blood test for biomarkers (the cardiac protein troponin).

Coronary artery bypass surgery, also known as coronary artery bypass grafting (CABG, pronounced “cabbage”) surgery, and colloquially heart bypass or bypass surgery, is a surgical procedure to restore normal blood flow to an obstructed coronary artery. A normal coronary artery transports blood to the heart muscle itself, not through the main circulatory system. There are two main approaches. In one, the left internal thoracic artery, LITA (also called left internal mammary artery, LIMA) is diverted to the left anterior descending branch of the left coronary artery. In this method, the artery is “pedicled” which means it is not detached from the origin. In the other, a great saphenous vein is removed from a leg; one end is attached to the aorta or one of its major branches, and the other end is attached to the obstructed artery immediately after the obstruction to restore blood flow. The obstruction being bypassed is typically due to arteriosclerosis, atherosclerosis, or both. Arteriosclerosis is characterized by thickening, loss of elasticity, and calcification of the arterial wall, most often resulting in a generalized narrowing in the affected coronary artery.

Myocarditis, also known as inflammatory cardiomyopathy, is inflammation of the heart muscle. Symptoms can include shortness of breath, chest pain, decreased ability to exercise, and an irregular heartbeat. The duration of problems can vary from hours to months. Complications may include heart failure due to dilated cardiomyopathy or cardiac arrest. Myocarditis is most often due to a viral infection. Other causes include bacterial infections, certain medications, toxins, and autoimmune disorders.

The terms single bypass, double bypass, triple bypass, quadruple bypass and quintuple bypass refer to the number of coronary arteries bypassed in the procedure. In other words, a double bypass means two coronary arteries are bypassed (e.g., the left anterior descending (LAD) coronary artery and right coronary artery (RCA)); a triple bypass means three vessels are bypassed (e.g., LAD, RCA and left circumflex artery (LCX)); a quadruple bypass means four vessels are bypassed (e.g., LAD, RCA, LCX and first diagonal artery of the LAD) while quintuple means five. Left main coronary artery obstruction requires two bypasses, one to the LAD and one to the LCX.

Peripheral Arteries carry blood away from the heart to the arms and legs of an individual.

### Cerebral Artery Bypass Surgery

A cerebral artery bypass procedure is much like a cardiac bypass procedure. But where a cardiac bypass procedure is done on an artery that feeds the heart, a cerebral artery bypass procedure is done on an artery in the brain. A cerebral artery bypass procedure uses a small artery from the scalp or a vein such as the saphenous vein from the leg to create a path or conduit around a blocked section of the cerebral artery such as the middle cerebral artery. The replacement blood vessel is connected or anastomosed to the narrowed artery beyond the occlusion first and then flushed with the antioxidant solution prior to the opposite end of the replacement vessel being anastomosed to a carotid artery or to an occipital artery. This inhibits reperfusion injury when blood-flow is restored to the ischemic tissue downstream from the blockage in the cerebral artery. This gives the section of the brain greater blood flow.

As used herein an antioxidant solution is comprised of a physiologic salt solution, which is ionically balanced, pH balanced to a physiologic pH, preferably 7.2-7.4, has at least one antioxidant and at least one substrate for nitric oxide synthase. In a preferred embodiment the solution contains ascorbic acid, glutathione and arginine as the substrate for nitric oxide synthase. A preferred physiological salt solution used to inhibit damage to the heart is comprised of a buffered salt solution, a sugar, ascorbic acid, glutathione and L-arginine. The buffered salt solution is comprised of a) calcium ions; b) potassium ions (from about 100 mM to about 250 mM; derived from compounds selected from the group consisting of potassium chloride, and potassium phosphate); c) magnesium ions (from about 2 mM to about 20 mM; derived from compounds selected from the group consisting of magnesium sulfate, and magnesium chloride); and d) sodium ions. To the buffered salt solution is added a sugar, preferably glucose, ascorbic acid, reduced glutathione and L-arginine to produce the physiological salt solution. In a preferred embodiment, the antioxidant solution contains D-glucose 1 g/L, calcium chloride (anhydrous) 0.14 g/L, potassium chloride 0.4 g/L, potassium phosphate 0.06 g/L, magnesium chloride. 6H₂O 0.1 g/L, magnesium chloride. 7 H₂O 0.1 g/L, sodium chloride 8 g/L, sodium bicarbonate 0.35 g/L, sodium phosphate 0.048 g/L, ascorbic acid, reduced glutathione, L-arginine, and optionally heparin. An example of the antioxidant/balanced salt solution used in the present invention is shown in the following Table. See U.S Pat. No. 6,569,615. Also, creatine and/or carnitine can be added to the solution to aid in the production of ATP in the mitochondria of the cells.

TABLE
Antioxidant Solution for the Prevention of Reperfusion Injury

| | *Concentration (±5%) | | |
|---|---|---|---|
| | | g/250 | |
| Components | g/L | mL | mM |
| Calcium chloride dihydrate | 0.14 | 0.0348 | 1.26 |
| Potassium chloride | 0.40 | 0.0998 | 5.37 |
| Potassium phosphate monobasic | 0.06 | 0.0148 | 0.44 |
| Magnesium sulfate heptahydrate | 0.10 | 0.0248 | 0.41 |
| Magnesium chloride hexahydrate | 0.10 | 0.0248 | 0.49 |
| Sodium chloride | 8.00 | 1.9998 | 136.89 |
| Sodium bicarbonate | 0.36 | 0.0900 | 4.17 |
| Sodium phosphate dibasic anhydrous | 0.026 | 0.0065 | 0.187 |
| L-Glutathione | 0.31 | 0.0775 | 1.01 |
| D-Glucose | 1.00 | 0.2500 | 5.55 |
| L-Arginine | 0.15 | 0.0375 | 0.86 |
| L-Ascorbic acid | 0.09 | 0.0225 | 0.51 |

| | |
|---|---|
| pH range | 7.3 ± 0.4 |

Administering the solution to the heart. To administer the balanced salt solution to the heart so that the entire heart receives or is perfused with the balanced salt solution, the solution can be delivered through the ostia in the aortic root at the right coronary artery for the right side of the heart or at the left coronary artery. This can be done alone or in conjunction with angiography.

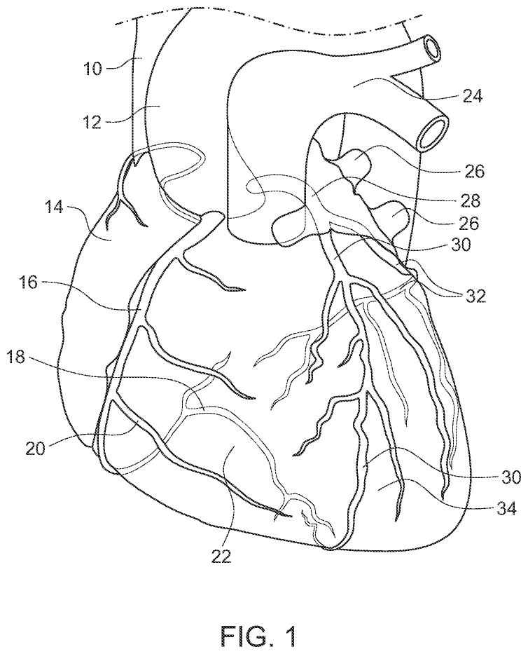

The Figures illustrate several embodiments of the present invention. FIG. 1 is a general schematic of a heart including the coronary arteries. Among the vessels illustrated are the superior vena cava 10, the aorta 12, the right atrium 14, the right coronary artery (RCA) 16, the posterior descending artery (PDA) 18, the right marginal artery (RMA) 20, the right ventricle 22, the left pulmonary artery 24, left pulmonary veins 26, the left coronary artery (LCA) 28, left anterior descending artery (LAD) 30, the circumflex artery 32 and the left ventricle 34.

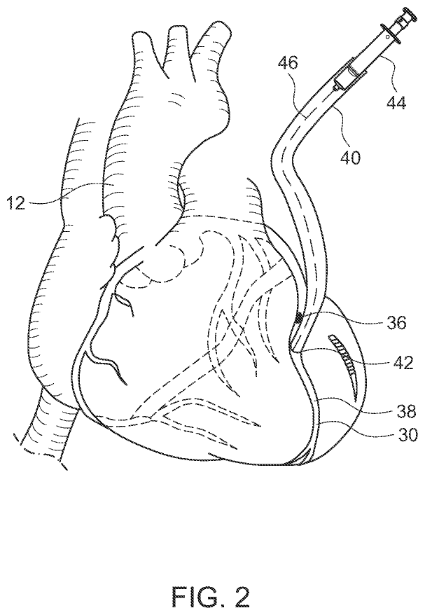

FIG. 2 shows the flushing of a section of a conduit such as the saphenous vein (40) the distal end of which (42) has been anastomosed to the left coronary artery (30) downstream from blockage/stenosis (36). Antioxidant/balanced salt solution 46 is being flushed using syringe 44 inserted at the proximal end of the saphenous vein 40. The distal end of saphenous vein 40 has been anastomosed to the left anterior descending artery (LAD) 30 at a section 38 downstream from the blockage 36 in the LAD 30. The antioxidant/balanced salt solution, 46, flushes through the saphenous vein 40 into section 38 of the left coronary artery 30. Other conduits that can be used include but are not limited to the internal thoracic artery, the radial artery, the right gastroepiploic artery, the ulnar artery the splenic artery and the inferior epigastric artery. In coronary artery bypass grafting (CABG) procedure, the solution can be used to flush the bypass conduit, generally a portion of a saphenous vein, to ascertain if there are any leaks in the conduit. For this purpose, a 10 mL syringe having a vessel tip is used to inject the solution. Generally, for CABG about 20 mL of the solution is used to flush an anastomosis, and no less than 10 mL. This is done for two purposes, one to check for leaks in the bypass conduits and the second is to administer the balanced salt solution to the area of the heart affected by the infarct to inhibit further damage to the heart.

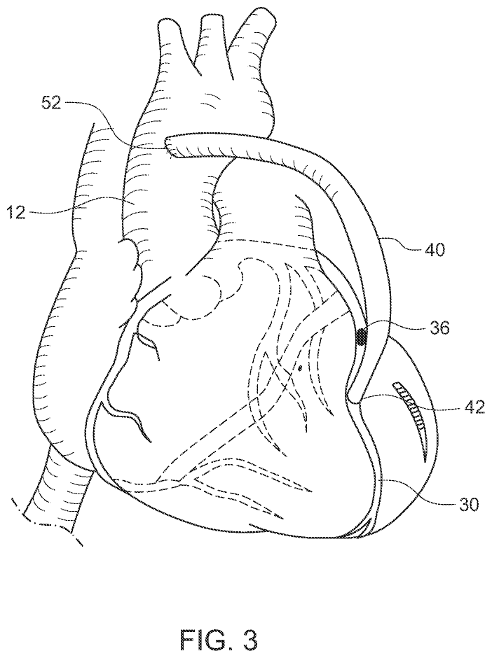

FIG. 3 shows the proximal end, 52, of conduit 40 anastomosed to aorta 12. As used herein, the end of a conduit anastomosed to a blood-providing vessel is termed the ‘proximal end’ of the conduit. The end of the conduit anastomosed to the end downstream from an occlusion is termed the ‘distal end’ of the conduit.

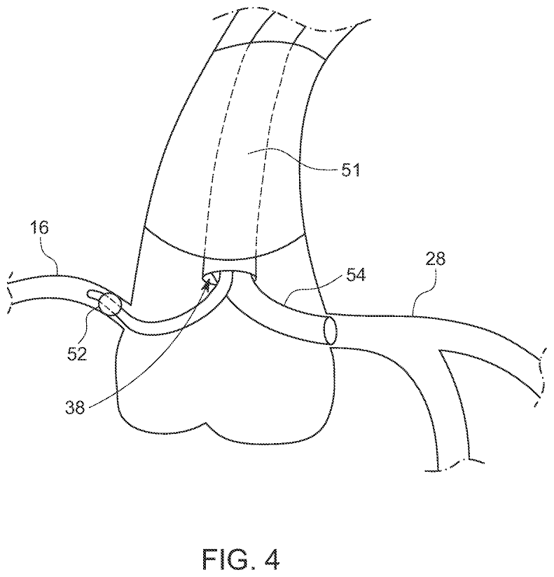

FIG. 4 illustrates a catheter, 51, threaded through the aorta. The catheter has two branches, 52, and 54. Branch 52 extends into the right coronary artery 16 and branch 54 extends through the left coronary artery 28. This allows the antioxidant solution to be infused into the coronary arteries and throughout all of the arteries of the heart before or after bypass surgery and before or after angioplasty or in conjunction with angiography. Percutaneous coronary intervention (PCI) such as percutaneous transluminal coronary angioplasty (PTCA) results in reperfusion injury as a result of toxic levels of ROS and inflammation being produced in the coronary artery as the artery is being opened and blood-flow is reintroduced. According to the present invention, the antioxidant solution is delivered to the portion of the artery distal from the occlusion within the artery prior to the occlusion being removed.

The following is a summary for the use of the antioxidant solution to prevent a reperfusion injury in ischemic tissue when the flow of blood is restored to the ischemic tissue in conjunction with angioplasty. If a coronary artery has an occlusion, the antioxidant solution can be delivered to the coronary artery distal from the occlusion prior to opening the stenotic region of the artery by angioplasty. Thus, the anti-ROS, and anti-free radical components in the antioxidant solution inhibit reperfusion injury that occurs when the flow of blood is re-introduced to the coronary artery distal from the occlusion. This method can be used not only for coronary angioplasty but also peripheral artery, venous, carotid artery, and renal artery angioplasty.

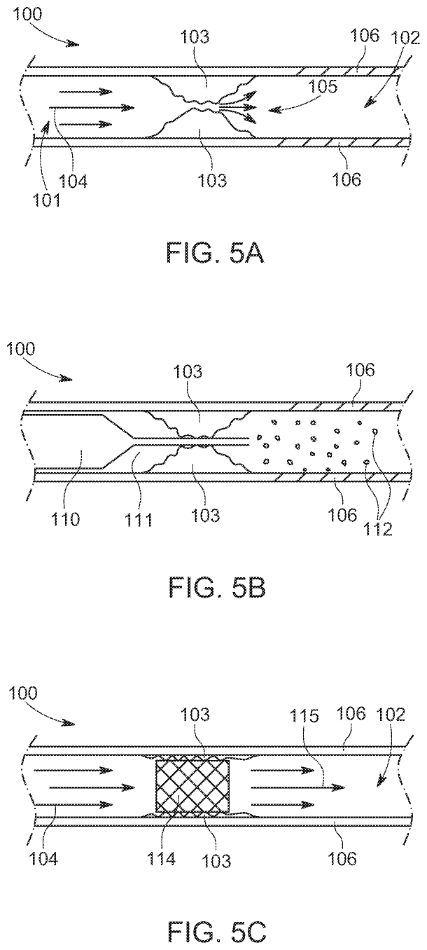

FIGS. 5A-5C illustrate this embodiment of the invention. FIGS. 5A-5C generally describe a catheter that can be used for delivering the antioxidant solution to an ischemic tissue to prevent reperfusion injury. FIG. 5A illustrates a cross-sectional view of an arterial vessel 100 whose blood flow is occluded by lesion 103 (i.e., plaque accumulation). The artery can potentially be in any part of the body. As shown, the relatively normal blood flow 104 near the region proximal 101 to lesion 103 is significantly blocked near the region distal 102 to lesion 103, producing very little blood flow 105 downstream to lesion 103. The lack of oxygenated blood to distal region 105 results in ischemic conditions, potentially resulting in a myocardial infarction in the case of a heart. One method to re-establish blood flow to distal region 102 may be the application of a reperfusion procedure such as angioplasty. In one embodiment, a dilatation device may be used to treat lesion 103, such as the application of a balloon catheter or a stent to lesion 103. According to the present invention, the ischemic region 106 is treated with an antioxidant solution to prevent reperfusion injury prior to the removal of the lesion. In particular, the antioxidant solution is allowed to be absorbed into the arterial walls of the ischemic region distal from the lesion prior to re-establishment of blood flow past lesion 103. To maximize the effectiveness the administration of the antioxidant solution to ischemic region 106, blood flow past lesion 103 is blocked. The treatment of ischemic region 106 with the antioxidant solution first involves occluding blood flow near the region proximal 101 to lesion 103. As illustrated in FIG. 5B, a balloon catheter 110 (or other occlusive device) may be inflated (or otherwise radially expanded) near the proximal region 101 to occlude blood flow 104. Balloon catheter 110 also includes an infusion catheter portion, basically a needle 111 that is advanced past lesion 103 to the region distal from the lesion. The infusion catheter 110 is used to deliver the antioxidant solution 112 to the ischemic region 106 of arterial vessel 100 prior to reperfusion of the portion of the artery downstream or distal from the lesion 103. The infusion catheter along with balloon catheter 110 is then retracted from arterial vessel 100, allowing for lesion 103 to be treated with a dilating device to re-establish blood flow to ischemic region 106. As illustrated in FIG. 5C, a balloon-expandable stent or a self-expanding stent may be deployed to lesion 103 to reperfuse arterial vessel 100 distal to lesion 103. For clarity, this embodiment, and other embodiments presented herein, are described with respect to the prevention of reperfusion injury to arterial vessels. It may be appreciated however, that in other embodiments, other vessels in the body (e.g., peripheral artery, veins, carotid artery, cerebral arteries and renal artery) impacted by lesions or other forms of blockage may be treated to prevent reperfusion injury with the apparatus and the antioxidant solution physiological salt solution described herein. For example, balloon catheter 110 may be used for the vessels affected by vulnerable plaque. See U.S. Pat. No. 7,837,650. In a preferred embodiment, antioxidant solution is flushed through the newly opened vessel for 2 to 3 days to eliminate the ROS and inflammatory products. An example of this is by using a catheter described in FIG. 4.

Peripheral Vascular Bypass: A peripheral vascular bypass is a procedure performed to reroute blood flow around a blocked or occluded artery. It does so by creating a new pathway for blood to flow using a conduit such as a synthetic graft (plastic tube) or a vein harvested from the patient such as a saphenous vein. Peripheral vascular bypass is named for the artery whose blood flow will be bypassed and the arteries that will receive the rerouted blood. The three common vascular bypass surgeries are: (1) aortobifemoral, which reroutes blood from the abdominal aorta to the two femoral arteries in the groin, (2) femoropopliteal, which reroutes blood from the femoral to the popliteal arteries above or below the knee, and (3) femorotibial, which reroutes blood between the femoral artery and the tibial artery. The antioxidant solution can then be used to inhibit reperfusion injury by infusing the antioxidant solution through the bypass conduit after the conduit has been anastomosed to the artery downstream or distal from the occlusion and prior to anastomoses of the conduit onto the blood providing artery. This infusion inhibits reperfusion injury of the ischemic tissue.

### EXAMPLE

The antioxidant solution described in the Table provides myocardial protection in Coronary Artery bypass grafting surgery. The purpose of the following example was to determine the impact of the antioxidant/balanced salt solution on myocardium during CABG procedures.

Material and Methods: The patients who were undergoing CABG surgery and taking part in the present study were divided into two groups, Group A containing 13 patients and Group B also containing 13 patients. The bypass conduits of Group A patients were flushed with the antioxidant/balanced salt solution having the composition depicted in the Table after anastomoses of the distal end of the conduit to the coronary artery but prior to anastomosis of the proximal end to the blood-providing vessel such as the aorta or branch of the aorta. The average amount of solution used to flush each conduit was 20 mL with a minimum of 10 mL

The bypass conduits of Group B patients were flushed after anastomoses of the distal end of the conduit to the coronary artery but prior to anastomosis of the proximal end to the blood-providing vessel such as the aorta or branch of the aorta with the standard-of-care solution Biseko® (Biotest Pharma GmbH, Dreieich, Germany in combination with 0.9% saline. Biseko® is a plasma preparation without isoagglutinins and coagulation factors. The average amount of solution used to flush each conduit was 20 mL with a minimum of 10 mL. The mean number of anastomoses per each patient for both groups of patients was 3.2+/−0.8.

Cardiac biomarkers Troponine T and Creatine kinase were evaluated as measurement of the effects of the different solutions on the myocardium. Normal Troponine T level is less than 100 ng/L. There are a number of different biomarkers used to determine the presence of cardiac muscle damage. Troponine T, measured through a blood test, is considered to be the best, and is preferred because it has greater sensitivity and specificity for measuring injury to the heart muscle than other tests. A rise in troponin occurs within 2-3 hours of injury to the heart muscle, and peaks within 1-2 days. The level of Troponin T, as well as a change over time of the levels of Troponin T, are useful in measuring and diagnosing or excluding myocardial infarctions, and the diagnostic accuracy of troponin testing is improving over time. One high-sensitivity cardiac troponin diagnostic is able to rule out a heart attack as long as the ECG is normal. Creatine kinase is not as specific as troponins for acute myocardial injury, and may be elevated with past cardiac surgery, inflammation or electrical cardioversion; it rises within 4-8 hours and returns to normal within 2-3 days.

### Results

### 12 Hours Post OP

Group A Patients: Troponin T levels averaged 2598 ng/L
Group B Patients: Troponin T levels 4128 ng/L

### 24 Hours Post OP

Group A Patients: Troponin T levels averaged 5666 ng/L
Group B Patients: Troponin T levels 13619 ng/L
Group A Patients: Creatine Kinase levels 483 U/L
Group B Patients: Tro Creatine Kinase levels 668 U/L

### 48 Hours Post OP

Group A Patients: Troponin T levels averaged 2655 ng/L
Group B Patients: Troponin T levels 2869 ng/L

### 72 Hours Post OP

Group A Patients: Troponin T levels averaged 1357 ng/L
Group B Patients: Troponin T levels 1530 ng/L

Conclusion: As can be seen from the data. The levels of Troponin T in the Group A patients were lower at every time point than the levels of the patients of Group B. This indicates that the antioxidant solution of the Table containing ascorbic acid, glutathione, glucose and L-arginine is beneficial in protecting or preventing reperfusion injury in the myocardium subsequent to coronary-bypass grafting surgery when compared to the standard of care 0.9% Saline/Biseko® (Biotest Pharma GmbH, Dreieich, Germany) preparations in “on-pump” CABG procedures.

What is claimed is:

1. A method for inhibiting reperfusion injury in a tissue, wherein the tissue has blood flow blocked by an occlusion within a vein or artery and wherein the vein or artery has an area upstream from the occlusion wherein the blood-flow is not occluded, and the vein or artery has an area downstream or distal from the occlusion wherein the blood-flow into the area is blocked by the occlusion comprised of:
administering an antioxidant solution into the vein or artery downstream from the occlusion prior or in conjunction to removing the occlusion and prior to re-establishing blood-flow to the tissue, wherein the antioxidant solution is comprised of water, dissolved salts of calcium, potassium, magnesium, sodium, one or more buffers, L-Arginine, glucose, glutathione, and ascorbic acid and wherein the solution is ionically balanced and pH balanced to a physiologic pH, preferably 7.2-7.4.

2. The method of claim 1 wherein the dissolved salts are comprised of calcium chloride dihydrate, potassium chloride, potassium phosphate monobasic, magnesium sulfate heptahydrate, magnesium chloride hexahydrate, sodium chloride sodium bicarbonate, and sodium phosphate dibasic anhydrous.

3. The method of claim 1 further comprising administering the antioxidant solution to an area within the vein or artery distal or downstream from the occlusion prior to removal of the occlusion and then removing the occlusion and re-establishing blood-flow to the area within the vein or artery distal to the lesion.

4. The method of claim 3 wherein the occlusion is removed from the vein or artery by means of angioplasty.

5. The methods of claims 1 wherein the artery having the occlusion is selected from the group consisting of a coronary artery, a peripheral artery, a carotid artery, a cerebral artery and a renal artery.

6. The method of claim 1 wherein the artery containing the occlusion is an artery of the heart selected from the group consisting of the left coronary artery (LCA), the left anterior descending (LAD) coronary artery, the left circumflex artery, the left marginal artery, a diagonal branch of a coronary artery, the right coronary artery (RCA), a posterior descending artery (PDA) and a right marginal artery (RMA).

7. The method of claim 1 wherein the antioxidant solution is further comprised of creatine and/or carnitine.

8. A method for inhibiting reperfusion injury in tissue downstream from an occlusion in a vein or artery undergoing bypass surgery wherein a conduit having a proximal and distal end is anastomosed at the distal end of the conduit to a vein or artery downstream or distal from the occlusion in the vein or artery and flushing the conduit with an antioxidant solution prior to anastomosing the conduit to a blood-providing blood vessel at a proximal end of the conduit, wherein the antioxidant solution is comprised of water, dissolved salts of calcium, potassium, magnesium, sodium, one or more buffers, and L-Arginine, glucose, glutathione, and ascorbic acid and wherein the solution is ionically and pH balanced to a physiologic pH, preferably 7.2-7.4.

9. The methods of claims 8 wherein the artery having the occlusion is selected from the group consisting of a coronary artery, a peripheral artery, a carotid artery, a cerebral artery and a renal artery.

10. The method of claim 8 wherein the conduit is a portion of a blood vessel selected from the group consisting of the internal thoracic artery, the saphenous vein, the radial artery, the right gastroepiploic artery, the ulnar artery the splenic artery and the inferior epigastric artery.

11. The method of claims 8 wherein the artery containing the occlusion is an artery of the heart selected from the group consisting of the left coronary artery (LCA), the left anterior descending (LAD) coronary artery, the left circumflex artery, the left marginal artery, a diagonal branch of a coronary artery, the right coronary artery (RCA), a posterior descending artery (PDA) and a right marginal artery (RMA).

12. The method of claim 8 wherein the dissolved salts are comprised of calcium chloride dihydrate, potassium chloride, potassium phosphate monobasic, magnesium sulfate heptahydrate, magnesium chloride hexahydrate, sodium chloride sodium bicarbonate, and sodium phosphate dibasic anhydrous.

13. The method of claim 8 wherein the occluded artery is a middle cerebral artery of a brain.

14. The method of claim 8 wherein the antioxidant solution is further comprised of creatine and/or carnitine.

15. A method for inhibiting reperfusion damage to a heart comprised of administering to either a right or a left coronary artery of a heart an antioxidant solution wherein the antioxidant solution is comprised of water, dissolved salts of calcium, potassium, magnesium, sodium, one or more buffers, and L-Arginine, glucose, glutathione, and ascorbic acid wherein the solution is ionically and pH balanced to a physiologic pH, preferably 7.2-7.4, with the proviso that the antioxidant solution is not administered as a cardioplegic solution.

16. The method of claim 15 wherein the dissolved salts are comprised of calcium chloride dihydrate, potassium chloride, potassium phosphate monobasic, magnesium sulfate heptahydrate, magnesium chloride hexahydrate, sodium chloride sodium bicarbonate, and sodium phosphate dibasic anhydrous.

17. The method of claim 15 wherein the antioxidant solution is further comprised of creatine and/or carnitine.
